(19) United States
(12) Patent Application Publication
(10) Pub. No.: US 2023/0225842 A1
(43) Pub. Date: Jul. 20, 2023

(54) DENTAL HAND-HELD DEVICE FOR CURING MATERIAL AND STIMULATING FLUORESCENCE

(71) Applicant: Ivoclar Vivadent AG, Schaan (LI)

(72) Inventors: Tatiana GLEBOVA, Buchs (LI); Alexander GROSSE-HONEBRINK, Arbon (CH); Bruno SENN, Gais (CH)

(21) Appl. No.: 18/156,283

(22) Filed: Jan. 18, 2023

(30) Foreign Application Priority Data
Jan. 20, 2022 (EP) 22152354.1

(51) Int. Cl. A61C 13/15; A61B 1/04; G01N 21/64

(52) U.S. Cl. CPC A61C 19/004; A61B 1/043; G01N 21/6447

(57) ABSTRACT

A dental hand-held device (100), including a curing device (101) for curing a dental restauration material (103) with light in a first wavelength range; and a fluorescence excitation device (105) for exciting fluorescence of dental substance (107) with light in a second wavelength range.

### CROSS-REFERENCE TO RELATED APPLICATIONS

This application claims priority to European patent application No. 22152354.1 filed on Jan. 20, 2022, which disclosure is incorporated herein by reference in its entirety.

### TECHNICAL FIELD

The present invention relates to a dental hand-held device for curing a light-curing substance with light in a first wavelength range and for exciting fluorescence of dental substance with light in a second wavelength range.

### BACKGROUND

Today, dental diagnoses are made using dental expertise in the visual assessment of the dental status as well as by means of radiographs. In addition to X-rays, there are now intraoral scanners and intraoral cameras that use long-wave (infrared) radiation to transilluminate the teeth. This method can be used to visualize approximal caries. A dentist can visually identify certain indications. However, it is often difficult to distinguish restoration materials and tooth structure by eye. There is currently no simple solution to this problem.

US 20120219924 and U.S. Pat. No. 6,325,623 are directed to dental devices and are hereby incorporated by reference in their entirety.

### SUMMARY

It is the aim of the present invention to provide a dental hand-held device for curing a light-curing substance, with which additionally specific locations in a dentition can be made visible.

According to a first aspect, this technical aim is solved by a dental hand-held device, comprising a curing device for curing a dental restoration material with light in a first wavelength range; and a fluorescence excitation device for exciting fluorescence of the tooth substance with light in a second wavelength range.

The dental hand-held device allows a user to easily identify restorations and caries in a cavity and distinguish them from healthy tooth structure. In addition, excitation of bacterial metabolites can be used to identify and confirm other diagnoses (plaque, calculus). The advantage of combining a curing device, such as a polymerization lamp, and a fluorescence device is that identification of carious areas can also be performed during, before or after curing of dental restauration material.

In a technically advantageous embodiment of the dental hand-held device, the dental hand-held device comprises an electronic control unit for controlling the curing device and the fluorescence excitation device. This achieves, for example, the technical advantage that the curing device and the fluorescence excitation device can be controlled flexibly and independently.

In another technically advantageous embodiment of the dental hand-held device, the electronic control unit is designed to activate the curing device and the fluorescence excitation device independently of each other. This achieves, for example, the technical advantage that the curing device and the fluorescence excitation device can be switched on and off as required.

In another technically advantageous embodiment of the dental hand-held device, the electronic control unit is designed to activate the curing device and the fluorescence excitation device at a predetermined time interval. This achieves, for example, the technical advantage that the inspection of a cured filling can be carried out automatically after curing.

In another technically advantageous embodiment of the dental hand-held device, the dental hand-held device comprises an electronic camera for recording fluorescent light. An optical filter may be arranged in front of the camera, for example to filter out blue light. The camera achieves the technical advantage, for example, that fluorescence images of carious areas can be stored.

In another technically advantageous embodiment of the dental hand-held device, the dental hand-held device comprises an electronic interface for transmitting data from the electronic camera to an external device. This provides the technical advantage, for example, that digital fluorescence images of carious areas can be processed and evaluated externally.

In another technically advantageous embodiment of the dental hand-held device, the dental hand-held device comprises a single light source which is designed to emit light in the first wavelength range and the second wavelength range. This achieves, for example, the technical advantage that the dental hand-held device can be manufactured with little effort.

In another technically advantageous embodiment of the dental hand-held device, the dental hand-held device comprises an optical filter for blocking the first wavelength range or the second wavelength range. This achieves, for example, the technical advantage that the wavelength range of the respective light can be adjusted with high precision.

In another technically advantageous embodiment of the dental hand-held device, a beam path can be deflected by the optical filter or the filter can be moved into the beam path. This achieves, for example, the technical advantage that the frequency of the respective light can be controlled.

In another technically advantageous embodiment of the dental hand-held device, the dental hand-held device comprises a movable mirror or an electronically controllable micromirror for deflecting the beam path through the optical filter. This achieves, for example, the technical advantage that the beam path can be deflected in a simple manner.

In another technically advantageous embodiment of the dental hand-held device, the filter comprises an edge filter or a high- pass filter. This achieves, for example, the technical advantage that an unused wavelength range can be effectively suppressed.

In another technically advantageous embodiment of the dental hand-held device, the dental hand-held device comprises a first light source for emitting light in the first wavelength range and a second light source for emitting light in the second wavelength range. This achieves, for example, the technical advantage that a specific light source can be used for each application purpose, which is independent of the other light source and energy can be saved.

In another technically advantageous embodiment of the dental hand-held device, an intensity of the light of the curing device and/or of the light of the fluorescence excitation device is adjustable. For this purpose, the dental hand-held device may comprise a controller by means of which the intensity of the light of the curing device and/or the light of the fluorescence excitation device can be adjusted independently of one another. However, the controller may also be formed by the electronic control unit. The control device or the regulator may, for example, control the electrical power of the respective light source. This provides, for example, the technical advantage that the dental hand-held device can be adapted to specific lighting conditions.

In another technically advantageous embodiment of the dental hand-held device, activation of the curing device and/or the fluorescence excitation device can be selected. For this purpose, an electronic switch can be arranged in the dental hand-held device by means of which the activation of the curing device and/or the fluorescence excitation device can be selected manually. This achieves the technical advantage, for example, that a user can excite fluorescence or cure the dental restoration material as required.

In another technically advantageous embodiment of the dental hand-held device, the first wavelength range is between 380 nm and 515 nm and the second wavelength range is between 380 nm and 415 nm. This provides, for example, the technical advantage that the dental restoration material can be cured efficiently and carious areas can be easily detected. The second wavelength range can prevent over-illuminating of the fluorescence.

### BRIEF DESCRIPTION OF THE DRAWINGS

Examples of embodiments of the invention are shown in the drawings and are described in more detail below.

The FIGS. show:

FIG. 1 a schematic view of a dental hand-held device;

FIG. 2 a spectrum of a light source of the dental hand-held device; and

FIG. 3 another spectrum of a light source of the dental hand- held device.

### DETAILED DESCRIPTION

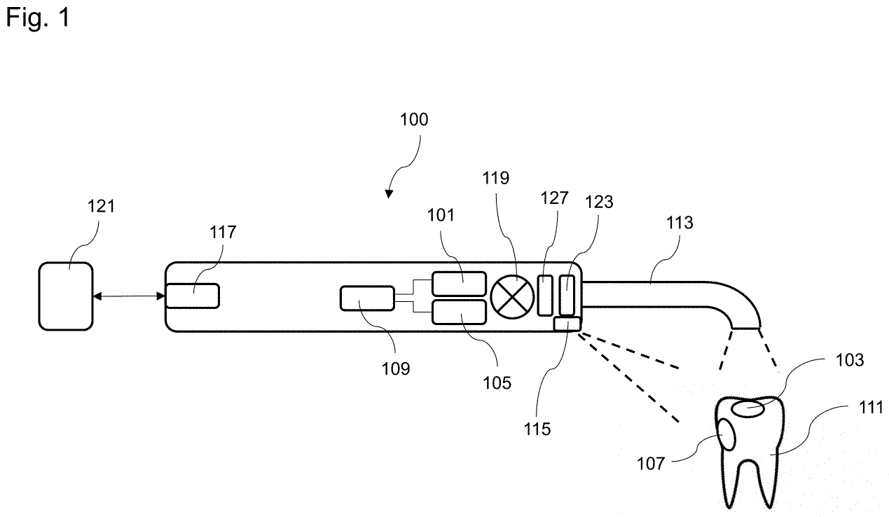

FIG. 1 shows a schematic view of a dental hand-held device 100. The dental hand-held device 100 is a portable electronic device that can be hand-held by a user during use. The dental hand-held device 100 includes a curing device 101 for curing a dental restoration material 103 with light in a first wavelength range. The light is emitted from a light source 119 within the dental hand-held device 100. The light source 119 is formed by, for example, a light emitting diode or a laser diode. The light is guided by a light guide 113 to a tooth 111 in which a dental restoration material 103 is disposed. The dental restoration material 103 is, for example, a polymer that is photo-inductively curable between 380-515 nm.

In addition to the curing device 101, the dental hand-held device 100 comprises a fluorescence excitation device 105 for exciting a fluorescence of carious tooth substance 107 of a tooth 111 with light in a second wavelength range. By the fluorescence excitation device 105, it is possible, for example, to excite a fluorescence of bacterial metabolites with blue light. A fluorescence that takes place is indicative of the presence of oral microorganisms and indicative of oral conditions, such as plaque, calculus, and caries. Fluorescence is the spontaneous emission of light shortly after a material is excited by light. In this process, the emitted photons are usually lower in energy than those previously absorbed. Curing of the dental restoration material 103 can occur in both wavelength ranges, while fluorescence excitation occurs only in the second wavelength range.

The curing device 101 and the fluorescence excitation device 105 are controlled by an electronic control unit 109. The control device 109 is adapted to activate the curing device 101 and the fluorescence excitation device 105 independently of each other, or to activate them at a predetermined time interval, such as 3 seconds. This allows, for example, the fluorescence excitation device 105 to be activated after a predetermined period of time after the curing device 101 is deactivated. This can ensure that after the dental restoration material 103 has been cured at a carious site, it is always checked to see if there are any other carious sites. The control unit 109 may comprise, but is not limited to, for example, a microprocessor and a digital memory in which control programs and data are stored.

In addition, the dental hand-held device 100 may include an electronic camera 115 capable of obtaining a digital image of the fluorescent light generated by the tooth 111, such as a CCD camera. The electronic camera 115 may also be activated by the electronic control device 109. Through the digital recording, any fluorescence that occurs can be permanently documented as an image file. The image file can then be transmitted to an external device 121 via an electronic data interface 117. The data interface is formed, for example, by a WLAN or Bluetooth interface. At the external device 121, such as a computer, the digital image can be analyzed and exported to a medical record.

The dental hand-held device 100 may include a broadband light source 119 that outputs light having both the first wavelength range and the second wavelength range. However, the dental hand-held device 100 may also comprise two separate narrow band light sources 119, one of which outputs the first wavelength range and is associated with the curing device 101 and another of which outputs the second wavelength range and is associated with the fluorescence excitation device 105. These light sources can be turned on and off independently of each other.

However, during curing, two light sources 119 with different wavelength ranges may be turned on simultaneously, such as a light emitting diode with a wavelength of 410 nm and a light emitting diode with a wavelength of 465 nm, whereas during fluorescence excitation, only a single light source is turned on, such as the light emitting diode with a wavelength of 410 nm. Thus, multiple light sources 119 are turned on in the curing wavelength. Additionally, multiple light sources may be turned on as the light source 119 for a range of wavelengths, such as multiple light emitting diodes with a wavelength of 465 nm, to increase a power.

If only a single light source 119 is used, the dental hand-held device 100 includes an optical filter 123 for blocking the first wavelength range and/or the second wavelength range. The optical filter 123 is, for example, a bandpass filter or an edge filter.

Depending on the wavelength range used, the optical filter 123 may direct the path of light or move the optical filter 123 into the path of light. The control for this is performed by the electronic control unit 109.

This can be achieved by a mechanically movable mirror 127 or an array of electronically controllable micromirrors (DLP-Digital Light Processing) to redirect the beam path through the optical filter 123. Through the array of electronically controllable micromirrors, the light beam is split into pixels by a rectangular array of movable micromirrors and then reflected pixel-by-pixel either into or out of the projection path.

In addition, the blue light from the fluorescence excitation device 105 can excite greenish fluorescence from dental materials. Some dental materials used to fill carious lesions incorporate fluorescent pigments that can be excited to fluoresce in blue and ultraviolet light. This fluorescence makes dental restorations appear natural in sunlight and artificial ultraviolet light, as natural teeth also exhibit intrinsic fluorescence in this spectral range. The fluorescent offers simple possibility to make plaque, calculus and caries, but also dental restoration materials visible.

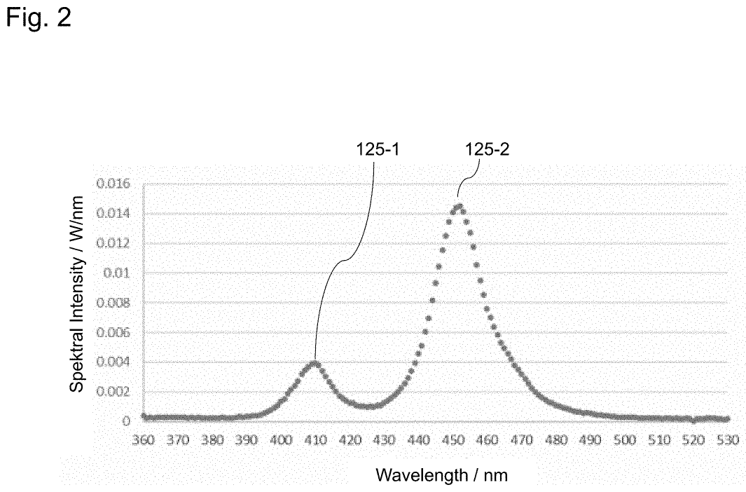

FIG. 2 shows a spectrum of a light source 119 of the dental hand-held device 100. The spectrum of the light source 119 has a first maximum 125-1 at a wavelength of 410 nm and a second maximum 125-2 at a wavelength of 450 nm.

To exclude over-radiation of the fluorescence, it is advantageous to excite only with short-wave light and block longer-wave light (450 nm). For example, to make fluorescence visible, the second maximum 125-2 is blocked by means of an optical filter 123. In this case, the dental hand-held device 100 emits only light of the first maximum 125-1 in the wavelength range between 380 nm and 415 nm.

Ideally, the fluorescence excitation device 105 emits at an excitation maximum of 405 nm (blue) and as narrow a bandwidth as possible. For example, the dental restoration material 103 fluoresces at a wavelength of 535 nm (green) and tooth substance 107 with plaque, calculus or caries fluoresces at a wavelength of 610 nm (red).

However, the dental hand-held device 100 may also have two different light sources 119, each emitting light in a different wavelength range. In this case, fluorescence can be made visible when only one light source is turned on to excite fluorescence and the other light source is turned off. The other light source is used to cure the dental restoration material 103.

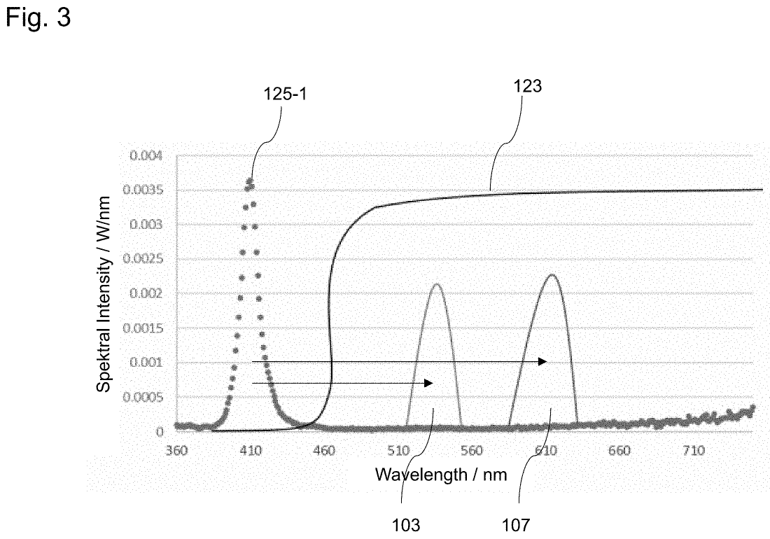

FIG. 3 shows another spectrum of a light source of the dental hand-held device 100. Fluorescence of tooth substance 107 in the green wavelength range (510 nm to 550 nm) or red wavelength range (580 nm to 640 nm) is excited by the light of the first maximum 125-1 in the wavelength range between 380 nm and 415 nm. Since the optical filter 123 blocks emission in wavelength range greater than 460 nm, the fluorescence can be readily detected. For example, the optical filter 123 is a high-pass filter.

In addition, a high-pass filter in the form of goggles or a shield can be used between a user and the patient to filter one wavelength and allow only longer wavelength fluorescence radiation to pass.

The dental hand-held device 100 enables the integration of the blue light-induced fluorescence diagnostic method in a dental polymerization lamp. The dental hand-held device 100 therefore combines various diagnostic options (fluorescence of dental restauration materials and bacterial metabolites) and treatment (curing of dental restauration materials) in a single device to support the dentist in the treatment process. The dental hand-held device 100 can also be used to detect residual calculus and plaque before and after dental hygiene treatment.

The dental hand-held device 100 facilitates a workflow of the dentist and a disinfection (avoidance of cross-contamination). Additional areas of application can be in tooth cleaning or caries diagnostics. The dental hand-held device 100 facilitates the possibility of diagnosis (caries, old filling) and enables treatment (polymerization lamp) in one unit. It allows to cover all steps of a filling placement with the same device (caries removal, removal of old fillings, curing of new fillings).

The red fluorescence of bacteria-generated dental indications, such as plaque, calculus or caries, can be efficiently visualized with the dental hand-held device 100. The dental hand-held device 100 thus offers a quick uncomplicated solution in a device that is already used by a dentist.

All features explained and shown in connection with individual embodiments of the invention may be provided in different combinations in the subject matter of the invention to simultaneously realize their beneficial effects.

All method steps can be implemented by means which are suitable for executing the respective method step. All functions that are executed by the objective features can be a method step of a method.

The scope of protection of the present invention is given by the claims and is not limited by the features explained in the description or shown in the figures.

### REFERENCE LIST

100 Dental hand-held device

101 Curing device

103 Dental restauration material

105 Fluorescence excitation device

107 Tooth structure

109 Electronic control unit

111 Tooth

113 Light guide

115 Electronic camera

117 Electronic data interface

119 Light source

121 External device

123 Optical filter

125 Maximum

127 Mirror/micromirror

1. A dental hand-held device (100), comprising
a curing device (101) for curing a dental restoration material (103) with light in a first wavelength range; and
a fluorescence excitation device (105) for exciting fluorescence of tooth substance (107) with light in a second wavelength range.

2. The dental hand-held device (100) according to claim 1, comprising
an electronic controller (109) for controlling the curing device (101) and the fluorescence excitation device (105).

3. The dental hand-held device (100) according to claim 2,
wherein the electronic controller (109) is configured to activate the curing device (101) and the fluorescence excitation device (105) independently of each other.

4. The dental hand-held device (100) according to claim 2,
wherein the electronic control unit (109) is adapted to activate the curing device (101) and the fluorescence excitation device (105) at a predetermined time interval.

5. The dental hand-held device (100) according to claim 1, comprising
an electronic camera (115) for capturing fluorescent light.

6. The dental hand-held device (100) according to claim 5, comprising
an electronic data interface (117) for transmitting data from the electronic camera (115) to an external device (121).

7. The dental hand-held device (100) according to claim 1, comprising
a light source (119) configured to emit light in the first wavelength range and the second wavelength range.

8. The dental hand-held device (100) according to claim 1, comprising
an optical filter (123) for blocking the first wavelength range and the second wavelength range.

9. The dental hand-held device (100) according to claim 8,
wherein a beam path through the optical filter (123) is deflectable or the filter (123) is movable into the beam path.

10. The dental hand-held device (100) according to claim 9, comprising
a movable mirror (127) or an electronically controllable micromirror (127) for redirecting the beam path through the optical filter (123).

11. The dental hand-held device (100) according to claim 8
wherein the filter (123) comprises an edge filter or a high pass filter.

12. The dental hand-held device (100) according to claim 1, comprising
a first light source for emitting light in the first wavelength range and a second light source for emitting light in the second wavelength range.

13. The dental hand-held device (100) according to claim 1,
wherein an intensity of the light of the curing device (101) and/or the light of the fluorescence excitation device (105) is adjustable.

14. The dental hand-held device (100) according to claim 1,
wherein activation of the curing device (101) and/or the fluorescence excitation device (105) is selectable.

15. The dental hand-held device (100) according to claim 1,
wherein the first wavelength range is between 380 nm and 515 nm and the second wavelength range is between 380 nm and 415 nm.
